(19) United States
(12) Patent Application Publication
(10) Pub. No.: US 2026/0126422 A1
(43) Pub. Date: May 7, 2026

(54) SYSTEM AND METHOD FOR REAL-TIME MONITORING OF PARTICLES IN OZONE

(71) Applicant: Finesse Technology Co., Ltd., Hsinchu County (TW)

(72) Inventors: SHIN-HUA TSENG, Hsinchu County (TW); YU-JUNG LIN, Hsinchu County (TW); YU-MING CHEN, Hsinchu County (TW)

(73) Assignee: Finesse Technology Co., Ltd., Hsinchu County (TW)

(21) Appl. No.: 19/043,499

(22) Filed: Feb. 2, 2025

(30) Foreign Application Priority Data
Nov. 4, 2024 (TW) 113211954
Jan. 3, 2025 (CN) 202510007486.X
Jan. 3, 2025 (TW) 114100301

(51) Int. Cl. G01N 33/00; G01N 15/14; G01N 15/1429

(52) U.S. Cl. CPC G01N 33/0039; G01N 15/1429; G01N 2015/1486; G01N 2015/1493

(57) ABSTRACT

Disclosed is a system and a method for real-time monitoring of particles in ozone comprising an ozone reduction device for heating and reducing an ozone provided by an ozone source into an oxygen along a spiral conveying path and a particle counter for real-time monitoring of particles in the oxygen. The ozone source optionally shunts to supply a portion of the ozone to the ozone reduction device and another portion of the ozone to a process equipment, the particle counter can be used to monitor particles in the oxygen in real time while the process equipment is performing a manufacturing process, thereby preventing other non-ozone particles contained in the ozone from causing pollution, and also proving that an ozone gas provided by an ozone generator as the ozone source or an ozone tail gas emitted by the process equipment as the ozone source does not contain polluting particles.

### CROSS-REFERENCE TO RELATED APPLICATION

This application claims the benefit of priority from, and is a continuation-in-part application of U.S. patent application Ser. No. 18/967,015, filed on Dec. 3, 2024, entitled “OZONE REDUCTION DEVICE”, which claims the benefit of priority of Taiwan Patent Application No. 113211954, filed on Nov. 4, 2024. In addition, this application also claims priority from Taiwan Patent Application No. 114100301, filed on Jan. 3, 2025; claims priority from Taiwan Patent Application No. 113211954, filed on Nov. 4, 2024; and claims China Patent Application No. 202510007486.X, filed on Jan. 3, 2025, each of which is hereby incorporated herein by reference in its entireties.

### BACKGROUND OF THE DISCLOSURE

### 1. Field of Disclosure

The disclosure relates to a system and a method for monitoring particles contained in ozone gas, more particularly to a method integrating ozone gas reduction technique and post-ozone reduction particle monitoring technique, and a system designed according to the method.

### 2. Related Art

Ozone has been found capable of oxidizing organic and/or metallic materials and thus can be applied in semiconductor wafer cleaning and processing, for example, to remove unwanted photoresist residues. Ozone can be used in gaseous form (dry ozone technique), but it can also be dissolved in water and used as ozone water (wet ozone technique). For example, ozone can be used to remove photoresist after a series of photolithography and etching processes. Dry ozone technique or wet ozone technique can be applied to the surface of semiconductor wafers. Dry ozone technique exposes the surface of semiconductor wafers to ozone gas and one type of gas or more than one type of gas to oxidize the materials on the surface of the wafer. Wet ozone technique exposes the semiconductor wafer surface to ozone and process fluid [such as deionized (DI) water or chemical solution] to oxidize the materials on the wafer surface.

Since the cleanliness of the wafer surface will affect the subsequent semiconductor manufacturing processes and the yield of products, to such an extent that up to 50% of all production losses are caused by wafer surface contamination. The most common major contamination includes metal, organic and particulate residues.

When ozone is used in semiconductor component manufacturing processes, contamination caused by impurities contained in ozone, especially metal contamination, is a serious problem. The metals constituting the contamination source include, for example, the metal electrodes of a reaction chamber where ozone is generated by high voltage discharge, or the reaction products resulting from the reaction between ozone and a metal pipeline used for supplying ozone. These metallic impurities have a significant impact on the performance of semiconductor components, including electrical properties such as electrical conductivity, resistance and dielectric constant. For example, metal contamination can cause leakage current in the p-n structure, which in turn leads to a decrease in the breakdown voltage of oxides and a reduction in the carrier life cycle.

The conventional technique known at present uses a gas filter to remove impurities from ozone used in semiconductor component manufacturing processes. One known conventional gas filter uses, for example, an adsorbent capable of adsorbing impurities to remove gaseous impurities. Another known conventional gas filter uses a filter material to filter impurities in the form of solid fine particles. In addition, conventional techniques known in the art have also attempted to continuously improve the electrode structure and electrode materials used for high voltage discharge in ozone generators, so as to make the generated ozone contain less metal impurities

Since ozone generators generate ozone by discharge between metal electrodes, metal particles generated by the metal electrodes are usually one of the sources of ozone pollution. In order to solve the above-mentioned problem of ozone pollution source, a conventional technique (e.g., Taiwan Patent Publication No. 200605208A) discloses adding a molecular permeable membrane capable of filtering metal particles into the ozone gas supply system. In addition, conventional techniques (e.g., U.S. Pat. Nos. 9,186,647B2 and 9,764,268B2) disclose adding a gas filter to an ozone generating device to filter solid particles with a particle size greater than 0.2 μm to remove impurities and foreign body. However, after using the gas filters or molecular permeable membranes of these ozone generating devices for a period of time, it is impossible to know whether they still maintain the expected effect. Usually, it is required to wait until the device is shut down and use a test wafer (blank wafer) and an optical microscope to perform a scan inspection.

In addition to cleaning, ozone has also been found capable of growing an oxide layer that can be used as a passivation layer or an interface layer for semiconductor components. Because ozone has extremely poor stability and can decompose into oxygen at room temperature, ozone cannot be stored. It is usually produced on-site using an ozone generator and used immediately. However, ozone is a gas that is harmful to both the human body and the environment. Although it can be decomposed into oxygen in the natural environment, this natural decomposition is very slow, so the ozone exhaust gas requires further treatment before it can be discharged. Furthermore, the prior art cannot prove whether the ozone tail gas emitted by an ozone source (e.g., semiconductor manufacturing process equipment) contains polluting particles. Although ozone reduction technique is currently available that can decompose ozone into oxygen, the half-life of ozone at 20 degrees Celsius is about 3 days, and the half-life decreases as the temperature increases. In order to completely reduce ozone to oxygen, the conventional technique requires a relatively high temperature (about 420 degrees Celsius or above) to achieve this effect. Furthermore, the conventional ozone reduction chamber is in a straight cylindrical shape, so the time for the ozone gas to pass through the straight cylindrical ozone reduction chamber is quite short. Furthermore, the conventional ozone reduction technique enables ozone to come into direct contact with the heating element, which can lead to corrosion of the heating element.

In summary, taking the semiconductor manufacturing process as an example, since the entire semiconductor manufacturing process usually takes more than a month from wafer loading to completion, if there is a loss, it will be in the billions of US dollars. Therefore, how to prevent losses or stop losses immediately is an object of all detection techniques, is also a target which the semiconductor industry has been striving for.

### SUMMARY OF THE DISCLOSURE

In view of this, one object of the disclosure is to provide a system and a method for real-time monitoring of particles in ozone, which are based on combination of an ozone reduction technique of an ozone reduction device and a particle (micronic dust or microparticle) detection technique of a particle counter (or particle size counter) capable of providing real-time monitoring of particle pollution concentration in an ozone gas and tracking particle types to analyze pollution sources, thereby solving the problems of the above-mentioned conventional techniques.

In order to achieve the above object, the disclosure discloses a system for real-time monitoring of particles in ozone, comprising: an ozone reduction device for heating and reducing an ozone provided by an ozone source into an oxygen along a spiral conveying path; and a particle counter for real-time monitoring of a number of a particle and/or a numerical value of a particle size in the oxygen.

Preferably, the ozone reduction device comprises: an air inlet conduit communicated to the ozone source; a gas conveying pipe used for introducing the ozone provided by the ozone source through the air inlet conduit, and the gas conveying pipe conveys the ozone via the spiral conveying path; a heating element used for providing a heat energy to heat the ozone conveyed by the gas conveying pipe, so that the ozone is heated by the heat energy when flowing along the spiral conveying path and reduced into the oxygen; and an air outlet conduit communicated to the gas conveying pipe to discharge the oxygen obtained by reducing the ozone.

Preferably, the gas conveying pipe is a spiral pipe, and the gas conveying pipe is spirally sleeved on an exterior of the heating element.

Preferably, the heating element heats only the ozone in the gas conveying pipe directly, heats the gas conveying pipe and the ozone in the gas conveying pipe simultaneously, and/or heats the ozone in the gas conveying pipe indirectly by heating the gas conveying pipe.

Preferably, the system for real-time monitoring of particles in ozone of the disclosure further comprises a heat insulation element, the heat insulation element coating one of or more than one of the gas conveying pipe, the heating element, the air inlet conduit and/or the air outlet conduit to maintain a heating temperature of the ozone.

Preferably, the system for real-time monitoring of particles in ozone of the disclosure further comprises a thermometer used for measuring a heating temperature of the heat energy provided by the heating element on the ozone in the gas conveying pipe.

Preferably, the system for real-time monitoring of particles in ozone of the disclosure further comprises a temperature control element used for controlling the heating element to provide the heat energy according to the heating temperature measured by the thermometer so as to heat the ozone to a preset temperature.

Preferably, the system for real-time monitoring of particles in ozone of the disclosure further comprises an air inlet adapter and an air outlet adapter, the air inlet adapter being connected between the air inlet conduit and the gas conveying pipe, the air outlet adapter being connected between the gas conveying pipe and the air outlet conduit.

Preferably, the system for real-time monitoring of particles in ozone of the disclosure further comprises a cooling device for cooling the oxygen obtained by heating and reducing the ozone with the ozone reduction device.

Preferably, the system for real-time monitoring of particles in ozone of the disclosure further comprises a process equipment, wherein the ozone source provides at least one portion of the ozone to the ozone reduction device for heating and reducing the at least one portion of the ozone into the oxygen, and the ozone source provides another portion of the ozone to the process equipment to perform a process step.

Preferably, the particle counter simultaneously and instantaneously monitors the number of the particle and/or the numerical value of the particle size in the oxygen generated by heating and reducing the at least one portion of the ozone when the process equipment uses the other portion of the ozone to perform the process step.

Preferably, the process equipment determines whether the ozone is contaminated by the particle based on the number of the particle and/or the numerical value of the particle size of the particle counter, thereby controlling to continue or to stop introducing the other portion of the ozone provided by the ozone source into the process equipment.

Preferably, the ozone source shunts to supply the at least one portion of the ozone and the other portion of the ozone through a shunt pipe, thereby providing the at least one portion of the ozone to the ozone reduction device and providing the other portion of the ozone to the process equipment respectively.

Preferably, a control valve is provided between the ozone source and the shunt pipe to control supplying or stop supplying the at least one portion of the ozone and/or the other portion of the ozone according to the number of the particle and/or the numerical value of the particle size.

Preferably, the particle counter uses a light source to provide a light ray to illuminate the oxygen, causing the particle in the oxygen to scatter or diffract, and then analyzes characteristics of the light ray of the light source to obtain the number of the particle and/or the numerical value of the particle size.

Preferably, the system for real-time monitoring of particles in ozone of the disclosure further comprises a pure oxygen source for supplying a pure oxygen to the ozone reduction device before the ozone reduction device heating the ozone provided by the ozone source to reduce the ozone into the oxygen until the number of the particle and/or the numerical value of the particle size monitored by the particle counter are/is zero.

In order to achieve the above object, the disclosure further discloses a method for real-time monitoring of particles in ozone, using the aforementioned system for real-time monitoring of particles in ozone to real-time monitor the particle in the ozone, comprising following steps: performing an ozone providing step, for providing the ozone using the ozone source; performing an oxidation-reduction step, for using the ozone reduction device to heat and reduce the ozone provided by the ozone source into the oxygen along the spiral conveying path; and performing a monitoring step, for using the particle counter to monitor in real time the number of the particle and/or the numerical value of the particle size in the oxygen.

Preferably, the method for real-time monitoring of particles in ozone of the disclosure further comprises performing a zeroing step after performing the ozone providing step and before performing the oxidation-reduction step, so as to make the number of the particle and/or the numerical value of the particle size obtained by monitoring with the particle counter zero.

Preferably, the method for real-time monitoring of particles in ozone of the disclosure further comprises performing a cooling step after performing the oxidation-reduction step and before performing the monitoring step, for cooling the oxygen obtained by heating and reducing the ozone with the ozone reduction device.

Preferably, the method for real-time monitoring of particles in ozone of the disclosure further comprises performing a shunt step for shunting supply of the ozone after performing the ozone providing step and before performing the oxidation-reduction step.

As described above, the system and the method for real-time monitoring of particles in ozone of the disclosure have one following advantage or more than one of following advantages.
(1) The disclosure can be used to prevent other non-ozone particles contained in an ozone from contaminating semiconductor wafers, and to prove that an ozone gas provided by an ozone source (e.g., ozone generator) or an ozone tail gas emitted by the ozone source (e.g., semiconductor manufacturing process equipment) does not contain polluting particles.
(2) A filtering effect of a gas filter configured for the ozone source (e.g., a gas inlet and a gas outlet of the ozone generator) can be instantaneously known, for example, whether the gas filter still has the filtering effect of removing impurities and foreign matter after a period of use can be instantaneously known.
(3) By combining the ozone reduction technique of the ozone reduction device and the particle detection technique of the particle counter, a size and a number of particles contained in the ozone gas can be monitored in real time, capable of monitoring a pollution concentration in real time and tracking particle types to analyze pollution sources.
(4) Using a spiral gas conveying pipe, such as a spiral quartz pipe, as the ozone reduction chamber that occupies less space than the conventional straight ozone reduction chamber and increases a heat transfer area can ensure that ozone molecules that flow into the spiral gas conveying pipe have enough heating time to make the ozone gas quickly reduce to oxygen, so it is very suitable for large-flow ozone gas reduction.
(5) By spirally sleeving the gas conveying pipe around an exterior of a heating element to locate the heating element inside a spiral interior of the spiral gas conveying pipe can provide better heating efficiency than the conventional techniques, thereby achieving an efficacy of reducing costs. It can also avoid the problem of direct contact of ozone with the heating element and the particle counter in the conventional techniques, which leads to corrosion of the heating element and damage of the particle counter.
(6) A cooling device can be used to cool an oxygen obtained by heating and reducing ozone to a suitable temperature before the oxygen enters the particle counter.

In order to enable the examiner to have a further understanding and recognition of the technical features of the disclosure, preferred embodiments in conjunction with detailed explanation are provided as follows.

### BRIEF DESCRIPTION OF THE DRAWINGS

FIG. 1 is a schematic diagram of a first embodiment of a system for real-time monitoring of particles in ozone of the disclosure.

FIG. 2I and FIG. 2II are perspective diagrams of combination of a gas conveying pipe and a heating element of an ozone reduction device shown in FIG. 1, wherein FIG. 2I is a perspective diagram before combination, and FIG. 2II is a perspective diagram after combination.

FIG. 3 is a schematic diagram of an ozone reduction experimental device used in the system for real-time monitoring of particles in ozone of the disclosure.

FIG. 4 is a 10-second measurement chart of an ozone reduction experiment of the system for real-time monitoring of particles in ozone of the disclosure.

FIG. 5 is a 30-second measurement chart of an ozone reduction experiment of the system for real-time monitoring of particles in ozone of the disclosure.

FIG. 6 is a schematic diagram of a first embodiment of a method for real-time monitoring of particles in ozone of the disclosure.

FIG. 7 is a schematic diagram of a second embodiment of the system for real-time monitoring of particles in ozone of the disclosure.

FIG. 8 is a schematic diagram of a second embodiment of the method for real-time monitoring of particles in ozone of the disclosure.

### DETAILED DESCRIPTION OF THE DISCLOSURE

In order to understand the technical features, content and advantages of the disclosure and its achievable efficacies, the disclosure is described below in detail in conjunction with the figures, and in the form of embodiments, the figures used herein are only for a purpose of schematically supplementing the specification, and may not be true proportions and precise configurations after implementation of the disclosure; and therefore, relationship between the proportions and configurations of the attached figures should not be interpreted to limit the scope of the claims of the disclosure in actual implementation. In addition, in order to facilitate understanding, the same elements in the following embodiments are indicated by the same referenced numbers. And the size and proportions of the components shown in the drawings are for the purpose of explaining the components and their structures only and are not intending to be limiting.

Unless otherwise noted, all terms used in the whole descriptions and claims shall have their common meaning in the related field in the descriptions disclosed herein and in other special descriptions. Some terms used to describe in the present disclosure will be defined below or in other parts of the descriptions as an extra guidance for those skilled in the art to understand the descriptions of the present disclosure.

The terms such as “first”, “second”, “third” and “fourth” used in the descriptions are not indicating an order or sequence, and are not intending to limit the scope of the present disclosure. They are used only for differentiation of components or operations described by the same terms.

Moreover, the terms “comprising”, “including”, “having”, and “with” used in the descriptions are all open terms and have the meaning of “comprising but not limited to”.

A system and a method for real-time monitoring of particles in ozone of the disclosure combine an ozone reduction technique of an ozone reduction device and a particle detection technique of a particle counter, wherein the ozone reduction technique first fully heats an ozone provided by an ozone source through a spiral conveying path to reduce the ozone into an oxygen, and then the oxygen enters the particle counter for particle detection.

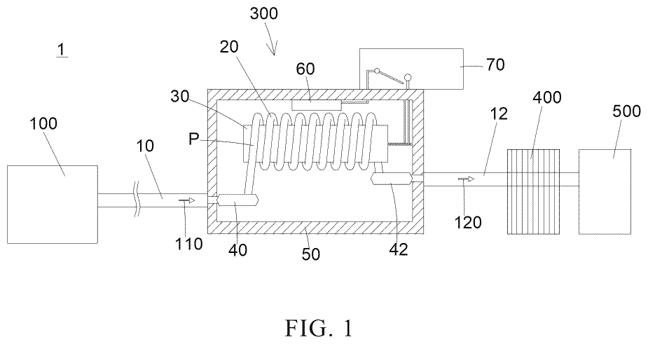

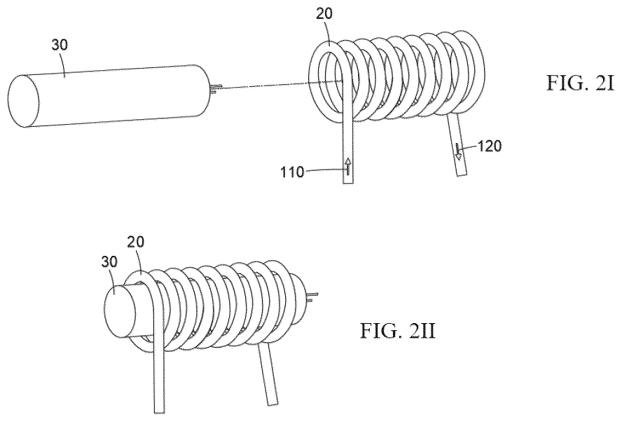

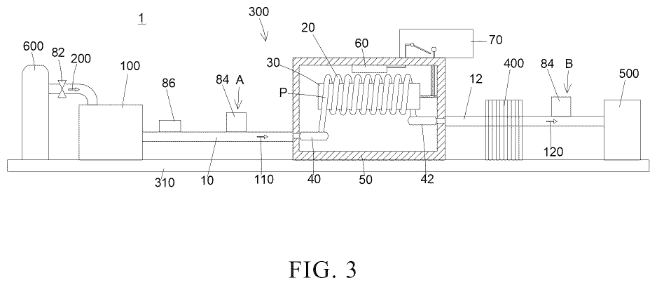

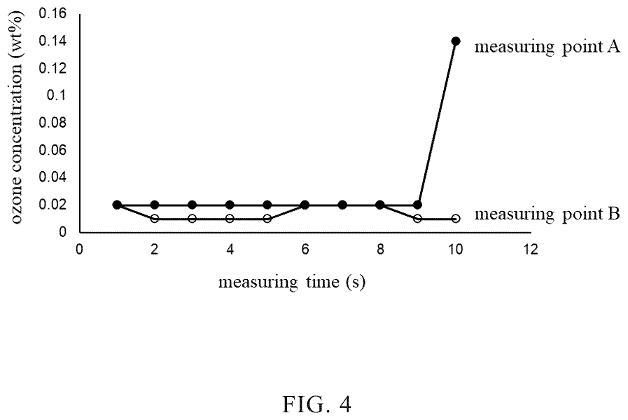

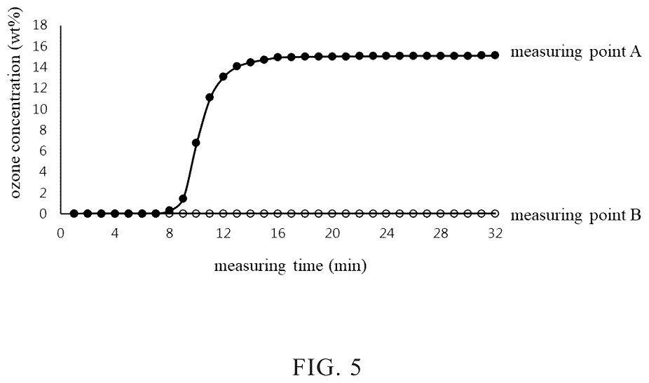

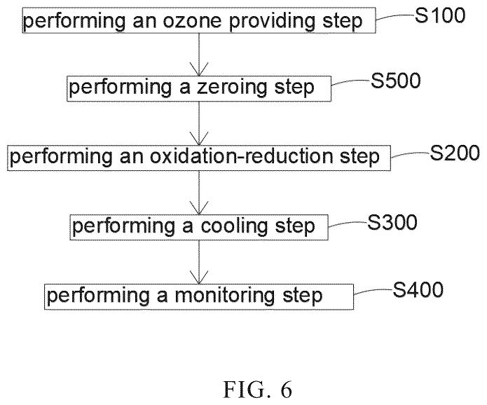

Please refer to FIG. 1 to FIG. 6. FIG. 1 is a schematic diagram of a first embodiment of a system for real-time monitoring of particles in ozone of the disclosure. FIG. 2 I and FIG. 2II are perspective diagrams of combination of a gas conveying pipe and a heating element of an ozone reduction device shown in FIG. 1, wherein FIG. 21 is a perspective diagram before combination, and FIG. 2II is a perspective diagram after combination. FIG. 3 is a schematic diagram of an ozone reduction experimental device used in the system for real-time monitoring of particles in ozone of the disclosure. FIG. 4 is a 10-second measurement chart of an ozone reduction experiment of the system for real-time monitoring of particles in ozone of the disclosure. FIG. 5 is a 30-second measurement chart of an ozone reduction experiment of the system for real-time monitoring of particles in ozone of the disclosure. FIG. 6 is a schematic diagram of a first embodiment of a method for real-time monitoring of particles in ozone of the disclosure.

As shown in FIG. 1 to FIG. 6, a first embodiment of a system 1 for real-time monitoring of particles in ozone of the disclosure comprises an ozone reduction device 300 and a particle counter 500. The method for real-time monitoring of particles in ozone of the disclosure comprises following steps: performing an ozone providing step S100, for providing an ozone 110 using an ozone source 100; performing an oxidation-reduction step S200, for using the ozone reduction device 300 to heat and reduce the ozone 110 provided by the ozone source 100 in step S100 into an oxygen 120 along a spiral conveying path P, so that all of the ozone 110 conveyed along the spiral conveying path P has enough time to be heated and can be quickly reduced into the oxygen 120; and performing a monitoring step S400, for using the particle counter 500 to monitor in real time a number of a particle and/or a numerical value of a particle size in the oxygen 120 obtained by heating and reducing the ozone 110 by the ozone reduction device 300. The disclosure is capable of quickly and fully heating and reducing all of the ozone 110 entering the ozone reduction device 300 into the oxygen 120, and capable of monitoring particle pollution concentration contained in the ozone 110 supplied by the ozone source 100 in real time and tracking particle types to analyze pollution sources, for example, the disclosure can be used to instantaneously know whether a gas filter configured for the ozone source 100 (e.g., a gas inlet and an air outlet of an ozone generator) still has a filtering effect of removing impurities and foreign matter after being used for a period of time, and to prevent other non-ozone particles contained in the ozone 110 from contaminating semiconductor wafers, and to prove that an ozone gas provided by an the ozone source 100 (e.g., an ozone generator) or an ozone tail gas emitted by the ozone source 100 (e.g., semiconductor manufacturing process equipment) does not contain polluting particles.

The ozone reduction device 300 of the system 1 for real-time monitoring of particles in ozone of the disclosure is used to reduce the ozone 110 provided by the ozone source 100 into the oxygen 120. The ozone reduction device 300 of the disclosure comprises an air inlet conduit 10, a gas conveying pipe 20, a heating element 30 and an air outlet conduit 12. Two ends of the gas conveying pipe 20 are communicated to the air inlet conduit 10 and the air outlet conduit 12 respectively. The air inlet conduit 10 is communicated to the ozone source 100 for introducing the ozone 110 provided by the ozone source 100. The disclosure does not limit a type or a purpose of the ozone source 100. For example, the ozone 110 supplied by the ozone source 100 can be applied in semiconductor manufacturing processes such as cleaning a surface of a semiconductor wafer, so an amount of the ozone 110 introduced into the air inlet conduit 10 is, for example, only a portion of an ozone generation amount (or ozone supply amount) of the ozone source 100, and can even be, for example, the excess ozone 110 (i.e., not affecting operations of the semiconductor manufacturing processes) or the ozone 110 is provided according to monitoring requirements. The above applications are examples and are not intended to limit the disclosure.

The gas conveying pipe 20 of the ozone reduction device 300 introduces the ozone 110 provided by the ozone source 100 through the air inlet conduit 10. One of features of the disclosure is that the gas conveying pipe 20 has the spiral conveying path P, and the ozone 110 is conveyed along the spiral conveying path P. The heating element 30 provides a heat energy to heat the ozone 110 conveyed by the gas conveying pipe 20, so that the ozone 110 is heated by the heat energy provided by the heating element 30 when flowing along the spiral conveying path P (i.e., during a flow process) and reduced into the oxygen 120. In other words, the disclosure uses the gas conveying pipe 20 with a hollow spiral structure as an ozone reduction chamber, the ozone 110 can not only flow along the spiral conveying path P inside the gas conveying pipe 20 and perform the oxidation-reduction step S200, but also avoid the ozone 110 from contacting the heating element 30. The gas conveying pipe 20 is, for example, a hollow spiral pipe, such as a spiral quartz pipe, and the gas conveying pipe 20 is spirally disposed on the heating element 30, for example, sleeved on an exterior of the heating element 30, so that the heating element 30 is located in a spiral interior of the spiral gas conveying pipe 20. The gas conveying pipe 20 of the disclosure, for example, can comprise a hollow spiral pipe or can be composed of a hollow spiral pipe, so as to provide the spiral conveying path P mentioned above.

The heating element 30 of the disclosure is, for example, an electric heater such as a ceramic heating pipe, but is not limited thereto. The heating element 30 can also be, for example, any conventional heater, such as a resistive heater or a heat exchange heater. The disclosure uses a spiral quartz pipe to transport ozone gas, which can increase a contact area (i.e., heat transfer area) between the ozone 110 and a pipe wall of the spiral quartz pipe, and can ensure that gas molecules of the ozone 110 flowing into the spiral quartz pipe have sufficient heating time and can be sufficiently heated, so that the ozone 110 can be fully reduced into the oxygen 120 during a process of the ozone 110 flowing along the spiral conveying path P, that is, before the ozone 110 is led out of the spiral quartz pipe. The disclosure can quickly reduce the ozone 110 into the oxygen 120, and is therefore very suitable for large-flow ozone gas reduction. In addition, the disclosure is not limited to a specific method of heating the ozone 110. The heating element 30 of the disclosure can optionally heat only the ozone 110 directly, heat the gas conveying pipe 20 and the ozone 110 in the gas conveying pipe 20 simultaneously, and/or heat the ozone 110 in the gas conveying pipe 20 indirectly by heating the gas conveying pipe 20, which can be determined according to a material of the gas conveying pipe 20 and a heating type of the heating element 30.

The air outlet conduit 12 of the ozone reduction device 300 of the disclosure is communicated to the gas conveying pipe 20 to discharge the oxygen 120 obtained by reducing the ozone 110. The air outlet conduit 12 of the disclosure is not limited to a specific shape, and can be a straight pipe, a curved pipe, a spiral pipe, or a combination thereof or other forms.

In addition, the ozone reduction device 300 of the disclosure further optionally comprises a heat insulation element 50. A purpose of the heat insulation element 50 is to further ensure a uniform heating temperature to avoid rapid cooling. Therefore, the heat insulation element 50 can be optionally coated on any appropriate position and component, for example, coating one of or more than one of the gas conveying pipe 20, the heating element 30, the air inlet conduit 10 and/or the air outlet conduit 12 to maintain a temperature of heating the ozone 110, for example, an interior of the gas conveying pipe 20 is maintained at a preset temperature, wherein the preset temperature is, for example, a temperature that enables the ozone 110 to reduce into the oxygen 120. The heat insulation element 50 of the disclosure is, for example, but not limited to, ceramic fiber thermal insulation cotton, and the heat insulation element 50 is not limited to a specific size or specification, as long as thermal insulation and heat preservation effects can be provided, it falls within the scope of protection claimed by the disclosure. The preset temperature is, for example, 350 degrees Celsius, but the disclosure is not limited thereto. Since a half-life of the ozone 110 is inversely related to temperature, the preset temperature can be set, for example, to correspond to a length of the spiral conveying path P and/or a flow rate of the ozone 110. For example, when a length of the spiral conveying path P is about 276 cm, an inner diameter of the gas conveying pipe 20 is about 4 mm, and a diameter of a spiral structure is about 50 mm, when a flow rate of the ozone 110 is about 27 L/min, a residence time of the ozone 110 in the gas conveying pipe 20 is about 77 ms. In other words, as long as the ozone 110 is fully reduced into the oxygen 120 or a preset ratio of the ozone 110 is reduced into the oxygen 120 before the ozone 110 is led out of the ozone reduction device 300 of the disclosure, any specifications of the gas conveying pipe 20 and corresponding preset temperatures fall within the scope of protection claimed by the disclosure. The above-mentioned preset ratio can be determined according to actual requirements, and the disclosure is not limited to a specific numerical value. Furthermore, calculation of a residence time of the ozone 110 in the gas conveying pipe 20 is based on a conventional calculation formula of relationships between speed, distance and time, and thus it is not further described herein.

The ozone reduction device 300 of the disclosure can further optionally comprise a thermometer 60 and/or a temperature control element 70. The thermometer 60 is used to measure a heating temperature of the heat energy provided by the heating element 30 on the ozone 110 in the gas conveying pipe 20. The temperature control element 70 is used to control the heating element 30 to provide a heat energy to heat the ozone 110. For example, the temperature control element 70 is electrically connected to the thermometer 60 and the heating element 30 to control the heating element 30 to provide the heat energy according to the heating temperature measured by the thermometer 60 so as to heat the ozone 110 to the above-mentioned preset temperature. Wherein the thermometer 60 is, for example, placed above a middle section of the gas conveying pipe 20 to detect the heating temperature. The temperature control element 70 is, for example, located on an outer side of the heat insulation element 50 to control a heating temperature of the ozone 110. The thermometer 60 and the temperature control element 70 of the disclosure can be, for example, a conventional temperature sensor and a conventional temperature controller respectively.

The ozone reduction device 300 of the disclosure further optionally comprises an air inlet adapter 40 and an air outlet adapter 42. The air inlet adapter 40 is connected between the air inlet conduit 10 and the gas conveying pipe 20. The air outlet adapter 42 is connected between the gas conveying pipe 20 and the air outlet conduit 12. Structures of the air inlet adapter 40 and/or the air outlet adapter 42 are, for example, but not limited to, Teflon coated with stainless steel, for example, a Teflon layer is coated with a stainless steel layer.

The system 1 for real-time monitoring of particles in ozone of the disclosure further optionally comprises a cooling device 400. The method for real-time monitoring of particles in ozone of the disclosure further optionally comprises performing a cooling step S300 after performing the oxidation-reduction step S200 and before performing the monitoring step S400, for using the cooling device 400 to cool the oxygen 120 obtained by heating and reducing the ozone 110 with the ozone reduction device 300, for example, reducing a temperature of the oxygen 120 discharged from the gas conveying pipe 20. The cooling device 400 is, for example, disposed on the air outlet conduit 12 or between the gas conveying pipe 20 and the air outlet conduit 12. Wherein the cooling device 400 can be disposed at any position, as long as a cooling effect can be achieved, it falls within the scope of protection claimed by the disclosure. The cooling device 400 is, for example, but not limited to, an air-cooled, a liquid-cooled, a phase-change or a hybrid cooler. As long as a temperature of the oxygen 120 can be reduced, any type of the cooling device 400 falls within the scope of protection claimed by the disclosure. In addition, the disclosure can optionally use a suction element (e.g., a suction pump) (not shown in the figures) to draw the oxygen 120 obtained by reducing the ozone 110 by the ozone reduction device 300 into the cooling device 400 to provide a cooling effect. Wherein a disposing location of the suction element is not particularly limited, it can be, for example, disposed at any location on the air outlet conduit 12, as long as the oxygen 120 can be cooled by the cooling device 400, it falls within the scope of protection of the disclosure.

The system 1 for real-time monitoring of particles in ozone of the disclosure uses a particle counter 500 to real-time monitor a number of a particle and/or a numerical value of a particle size in the cooled oxygen 120, for example, real-time monitoring a pollution concentration of particles contained in the ozone 110 provided by the ozone source 100 and tracking particle types to analyze pollution sources. The particle counter 500 used in the disclosure is not limited to a specific type or an operating principle, as long as it can be used to detect particles, it belongs to the scope of protection claimed in the disclosure. The particle counter 500 can be, for example, a commercially available particle counter or a particle counter using any particle or particle size detection technique, thus it is not described herein. For example, the particle counter 500 provides a light source (e.g., a collimated light source) to illuminate the oxygen 120, causing suspended particles in the oxygen 120 to scatter or diffract, and then analyzes a size and a quantity of the suspended particles by analyzing characteristics of the light source. In detail, the particle counter 500 is an instrument for measuring a size and a concentration of particles in ozone. Its principle is to infer a size and a concentration of particles by detecting scattering and absorption of light by particles. When using the particle counter 500, for example, the oxygen 120 passes through a fine hole or channel and optical detection is performed to measure a size and a number of particles. Wherein there is no particular limitation on types or concentrations of particles that the particle counter 500 can detect.

The method for real-time monitoring of particles in ozone of the disclosure further optionally comprises performing a zeroing step S500 after performing the ozone providing step S100 and before performing the oxidation-reduction step S200, so as to make a reading value obtained by monitoring with the particle counter 500 zero. The system 1 for real-time monitoring of particles in ozone of the disclosure optionally comprises a pure oxygen source 600 for supplying a pure oxygen 200 to the ozone reduction device 300 during the zeroing step S500 until a reading value (such as a number of a particle and/or a numerical value of a particle size) obtained by monitoring with the particle counter 500 is zero, then the ozone reduction device 300 is used to heat the ozone 110 provided by the ozone source 100 and reduce the ozone 110 into the oxygen 120. For example, the disclosure can, for example, turn on the temperature control element 70 to enable the spiral gas conveying pipe 20 to reach a high temperature in advance (the thermometer 60, for example, shows about 550 degrees Celsius), and then turn on the pure oxygen source 600 (for example, a high-pressure liquid oxygen bottle) to provide the pure oxygen 200 to the ozone source 100 (e.g., an ozone generator), and a flow rate of the pure oxygen 200 is controlled by a mass flow controller 82 to be about 2.83 L/min. The pure oxygen 200 is introduced before the ozone 110 is introduced into the air inlet conduit 10 so that the pure oxygen 200 is continuously input into the air inlet conduit 10 until a 0.1 μm reading value (e.g., a number of a particle and/or a numerical value of a particle size) of the particle counter 500 is zero, indicating that the air inlet conduit 10 is free of particle contamination at this time. Then, the ozone source 100 (e.g., an ozone generator) is turned on to generate the ozone 110 that flows into the air inlet conduit 10, and a back pressure is controlled by a pressure controller 86 to be about 30 Psi (pound force per square inch). Then, an ozone concentration detector 84 detects that a concentration of the ozone 110 reaches about 230 g/Nm³, and the ozone 110 is enabled to enter the ozone reduction device 300 for performing the oxidation-reduction step S200, wherein when the ozone 110 flows through the spiral gas conveying pipe 20 at about 550 degrees Celsius, the ozone 110 can be heated and reduced into the oxygen 120. The reduced oxygen 120 enters the cooling device 400 for cooling, for example. Then, the cooled oxygen 120 enters the particle counter 500 for particle measurement, wherein a particle size measurement range comprises, for example, about 0.1 μm, 0.2 μm, 0.3 μm, 0.5 μm, 0.7 μm, and 1.0 μm, and each test is about 60 seconds, one experiment is tested for a total of about 360 seconds, and for example, an experiment is repeated twice.

Monitoring results of the particle counter 500 are shown in Table 1:8 particles were measured in a first experiment, and 6 particles were measured in a second experiment. Among them, there are 3 to 4 particles with a particle size of 0.2 μm (inclusive) or less. From experimental results in Table 1, it can be known that by combining the ozone reduction technique with the particle measurement technique, the disclosure is capable of monitoring particles in the ozone 110 to prevent other non-ozone particles contained in (or carried by) the ozone 110 to contaminate a target object (e.g., a surface of a semiconductor wafer 800).

TABLE 1
Particle measurement results.

| | 0.1 | 0.2 | 0.3 | 0.5 | 0.7 | 1.0 | Particle/ | Particle/ | Sum |
|---|---|---|---|---|---|---|---|---|---|
| Item | μm | μm | μm | μm | μm | μm | min | m³ | Particle |
| 1 | 0 | 4 | 1 | 2 | 1 | 0 | 0.021 | 7.42 | 8 |
| 2 | 1 | 2 | 2 | 1 | 0 | 0 | 0.014 | 4.94 | 6 |

Please refer to FIGS. 3 to 5. FIG. 3 is a schematic diagram of an ozone reduction experimental device used in the system for real-time monitoring of particles in ozone of the disclosure. FIG. 4 is a 10-second measurement chart of an ozone reduction experiment of the system for real-time monitoring of particles in ozone of the disclosure. FIG. 5 is a 30-second measurement chart of an ozone reduction experiment of the system for real-time monitoring of particles in ozone of the disclosure. The disclosure measures ozone concentrations at two sides of the gas conveying pipe 20 (e.g., measurement point A and measurement point B) respectively by means of the ozone concentration detector 84, and thus ozone reduction results of the ozone reduction device 300 of the disclosure can be obtained. For example, the disclosure takes the ozone source 100 as an ozone generator, the disclosure provides the pure oxygen 200 to the ozone source 100 (e.g., ozone generator) through the pure oxygen source 600 (e.g., a high-pressure liquid oxygen bottle), and the ozone 110 is provided by reacting the pure oxygen 200 into ozone gas. Furthermore, before the ozone 110 enters the gas conveying pipe 20 of the ozone reduction device 300, the temperature control element 70 is used to make the gas conveying pipe 20 reach a high temperature in advance, the thermometer 60 shows about 350 degrees Celsius, for example, and then the ozone 110 enters the gas conveying pipe 20 of the ozone reduction device 300 through the air inlet conduit 10. In detail, the ozone 110 enters the gas conveying pipe 20 through the air inlet adapter 40, a temperature of the gas conveying pipe 20 is about 350 degrees Celsius, after the ozone 110 flows through the gas conveying pipe 20 at 350 degrees Celsius, the ozone 110 enters the air outlet conduit 12 through the air outlet adapter 42. After the reduced gas passes through the cooling device 400, an ozone concentration (measurement point B) is measured again by the ozone concentration detector 84 and then discharged. The disclosure uses, for example, the mass flow controller 82 (or referred to as a mass and flow rate controller) to control a flow rate of the oxygen 200 to 27 L/min, and the pressure controller 86 to control a back pressure to 30 Psi (pound force per square inch). The ozone reduction device 300 of the disclosure is, for example, disposed on a workbench 310 to perform an ozone reduction experiment, but is not limited thereto.

Please refer to FIG. 4. It can be known from data shown in FIG. 4 that when an ozone concentration at the measurement point A increases rapidly from 0.02 wt % to 0.14 wt %, the ozone reduction device 300 of the disclosure is capable of reducing 0.14 wt % of ozone to 0.01 wt % (measurement point B) in less than one second.

Please refer to FIG. 5. FIG. 5 shows data of continuously performing ozone reduction reaction with the ozone reduction device 300 of the disclosure and simultaneously measuring ozone concentrations (measurement point A, measurement point B) for 30 minutes. It can be known from FIG. 5 that even after a long period of operation, the ozone reduction device 300 of the disclosure can still maintain a good reaction.

In the above ozone reduction experiment, a flow rate of the ozone 110 supplied by the ozone source 100 is about 27 liters per minute, and an ozone concentration is about 15.3 wt % (measurement point A). After conversion, it can be known that there are about 354 grams of ozone per hour, which are more than 3 times higher compared to an ozone concentration for general space disinfection and sterilization. The disclosure enables the ozone 110 to be heated for a sufficient time by using the gas conveying pipe 20 (e.g., spiral quartz pipe) and to be quickly reduced into the oxygen 120 (the ozone 110 is hardly detected at the measurement point B). Furthermore, the disclosure is capable of performing ozone reduction stably for a long period of time by using the heat insulation element 50 (e.g., a heat insulation material layer).

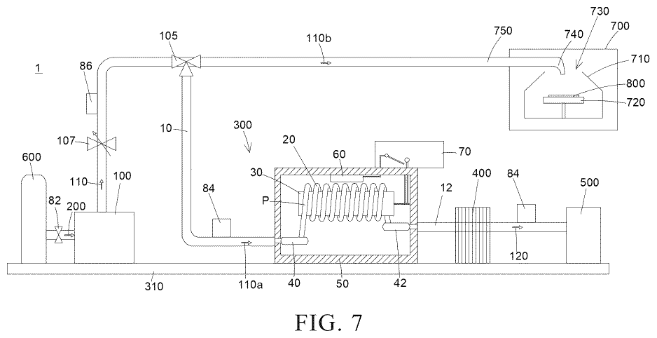

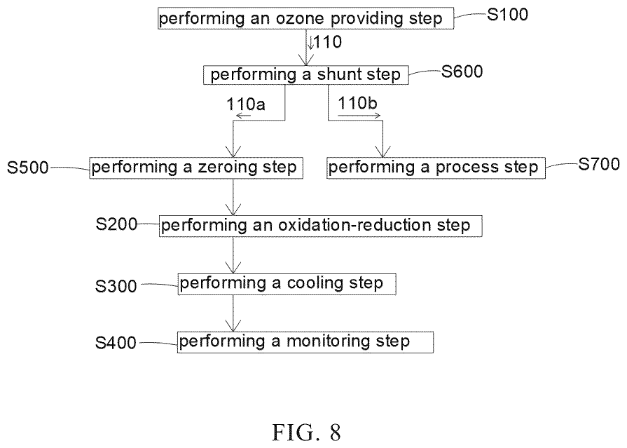

Please refer to FIG. 7 and FIG. 8, and please refer to FIG. 1 to FIG. 6 at the same time. FIG. 7 is a schematic diagram of a second embodiment of the system for real-time monitoring of particles in ozone of the disclosure. FIG. 8 is a schematic diagram of a second embodiment of the method for real-time monitoring of particles in ozone of the disclosure. Differences between the second embodiment and the first embodiment are that the system 1 for real-time monitoring of particles in ozone further comprises a process equipment 700 for performing a process step S700, and further comprises a shunt pipe 105 for performing a shunt step S600, for example, after the ozone source 100 provides the ozone 110, the shunt pipe 105 shunts at least one portion 110a of the ozone 110 provided by the ozone source 100 to the ozone reduction device 300, so as to heat and reduce the at least one portion 110a of the ozone 110 into the oxygen 120, and shunts another portion of ozone 110b of the ozone 110 provided by the ozone source 100 to the process equipment 700 for performing the process step S700. The process equipment 700 and the process step S700 mentioned above can be applied to, for example, but are not limited to, the field of semiconductor manufacturing. Regardless of any field, as long as particles in ozone gas are required to be monitored, the process equipment 700 and the process step S700 can be effectively applied to.

The disclosure can, for example, simultaneously and instantaneously perform the monitoring step S400 when the process equipment 700 performs the process step S700 to monitor a number of a particle and/or a numerical value of a particle size in the ozone 110, wherein the ozone source 100, for example, uses the shunt pipe 105 (e.g., a tee pipe) to perform the shunt step (S600) to shunt and supply the ozone 110 to the ozone reduction device 300 and the process equipment 700, for example, providing the at least one portion 110a of the ozone 110 and the other portion 110b of the ozone 110 to the ozone reduction device 300 and the process equipment 700 respectively. Thus, the particle counter 500 can simultaneously and instantaneously monitor a number of a particle and/or a numerical value of a particle size in the oxygen 120 generated by heating and reducing the at least one portion 110a of the ozone 110 when the process equipment 700 uses the other portion 110b of the ozone 110 to perform a process. Wherein the process equipment 700, for example, can optionally determine whether the ozone 110 is contaminated by particles based on a number of a particle and/or a numerical value of a particle size of the particle counter 500, thereby controlling to continue or to stop introducing the other portion 110b of the ozone 110 provided by the ozone source 100 into the process equipment 700. For example, a control valve 107 is optionally provided between the ozone source 100 and the shunt pipe 105 to control supplying or stop supplying the at least one portion 110a of the ozone 110 and/or the other portion 110b of the ozone 110 according to a number of a particle and/or a numerical value of a particle size.

In detail, taking the process equipment 700 as a semiconductor photoresist removal device as an example, the semiconductor photoresist removal device comprises a reaction chamber 710 and a carrier 720 capable of carrying a wafer 800 (or referred to as a target object). An upper opening 730 on the reaction chamber 710 is provided with a nozzle 740 connected to the ozone source 100 (e.g., an ozone generator) via a pipe 750 for supplying the ozone 110 to the wafer 800 (e.g., a semiconductor wafer) in the reaction chamber 710.

Wherein the pipe 750 connecting with the ozone source 100 (e.g., an ozone generator) and the nozzle 740 is provided with, for example, the control valve 107 and the shunt pipe 105 thereon. The shunt pipe 105 can shunt the ozone 110 to the ozone reduction device 300 via the air inlet conduit 10, and shunt the ozone 110 to the process equipment 700 via the pipe 750.

The ozone reduction device 300 of the disclosure, for example, reduces the ozone 110 into the oxygen 120 by means of the gas conveying pipe 20 (e.g., a spiral quartz pipe) and the heating element 30 (e.g., a ceramic heater), and the oxygen 120 is cooled by the cooling device 400 and then enters the particle counter 500. When the particle counter 500 detects particles, the process equipment 700 (e.g., semiconductor photoresist removal device) can quickly take appropriate measures, such as closing the control valve 107 to stop supply of the ozone 110, thereby the disclosure is capable of exerting effects of preventing losses from occurring or immediately stopping losses. Although the disclosure is described by taking the case of being applicable to semiconductor manufacturing as an example, the disclosure is not only applicable to the field of semiconductor manufacturing, but can be effectively applied to any field as long as it is desired to monitor particles in ozone gas.

Note that the specification relating to the above embodiments should be construed as exemplary rather than as limitative of the present disclosure, with many variations and modifications being readily attainable by a person of average skill in the art without departing from the spirit or scope thereof as defined by the appended claims and their legal equivalents.

As described above, the system and the method for real-time monitoring of particles in ozone of the disclosure have one following advantage or more than one of following advantages.
(1) The disclosure can be used to prevent other non-ozone particles contained in an ozone from contaminating semiconductor wafers, and to prove that an ozone gas provided by an ozone source (e.g., ozone generator) or an ozone tail gas emitted by the ozone source (e.g., semiconductor manufacturing process equipment) does not contain polluting particles.
(2) A filtering effect of a gas filter configured for the ozone source (e.g., a gas inlet and a gas outlet of the ozone generator) can be instantaneously known, for example, whether the gas filter still has the filtering effect of removing impurities and foreign matter after a period of use can be instantaneously known.
(3) By combining the ozone reduction technique of the ozone reduction device and the particle detection technique of the particle counter, a size and a number of particles contained in the ozone gas can be monitored in real time, capable of monitoring a pollution concentration in real time and tracking particle types to analyze pollution sources.
(4) Using a spiral gas conveying pipe, such as a spiral quartz pipe, as the ozone reduction chamber that occupies less space than the conventional straight ozone reduction chamber and increases a heat transfer area can ensure that ozone molecules that flow into the spiral gas conveying pipe have enough heating time to make the ozone gas quickly reduce to oxygen, so it is very suitable for large-flow ozone gas reduction.
(5) By spirally sleeving the gas conveying pipe around an exterior of a heating element to locate the heating element inside a spiral interior of the spiral gas conveying pipe can provide better heating efficiency than the conventional techniques, thereby achieving an efficacy of reducing costs. It can also avoid the problem of direct contact of ozone with the heating element and the particle counter in the conventional techniques, which leads to corrosion of the heating element and damage of the particle counter.
(6) A cooling device can be used to cool an oxygen obtained by heating and reducing ozone to a suitable temperature before the oxygen enters the particle counter.

Note that the specification relating to the above embodiments should be construed as exemplary rather than as limitative of the present disclosure, with many variations and modifications being readily attainable by a person of average skill in the art without departing from the spirit or scope thereof as defined by the appended claims and their legal equivalents.

What is claimed is:

1. A system for real-time monitoring of particles in ozone, comprising:
an ozone reduction device for heating and reducing an ozone provided by an ozone source into an oxygen along a spiral conveying path; and
a particle counter for real-time monitoring of a number of a particle and/or a numerical value of a particle size in the oxygen.

2. The system for real-time monitoring of particles in ozone as claimed in claim 1, wherein the ozone reduction device comprises:
an air inlet conduit communicated to the ozone source;
a gas conveying pipe used for introducing the ozone provided by the ozone source through the air inlet conduit, and the gas conveying pipe conveys the ozone via the spiral conveying path;
a heating element used for providing a heat energy to heat the ozone conveyed by the gas conveying pipe, so that the ozone is heated by the heat energy when flowing along the spiral conveying path and reduced into the oxygen; and
an air outlet conduit communicated to the gas conveying pipe to discharge the oxygen obtained by reducing the ozone.

3. The system for real-time monitoring of particles in ozone as claimed in claim 2, wherein the gas conveying pipe is a spiral pipe, and the gas conveying pipe is spirally sleeved on an exterior of the heating element.

4. The system for real-time monitoring of particles in ozone as claimed in claim 2, wherein the heating element heats only the ozone in the gas conveying pipe directly, heats the gas conveying pipe and the ozone in the gas conveying pipe simultaneously, and/or heats the ozone in the gas conveying pipe indirectly by heating the gas conveying pipe.

5. The system for real-time monitoring of particles in ozone as claimed in claim 2, further comprising a heat insulation element, the heat insulation element coating one of or more than one of the gas conveying pipe, the heating element, the air inlet conduit and/or the air outlet conduit to maintain a heating temperature of the ozone.

6. The system for real-time monitoring of particles in ozone as claimed in claim 2, further comprising a thermometer used for measuring a heating temperature of the heat energy provided by the heating element on the ozone in the gas conveying pipe.

7. The system for real-time monitoring of particles in ozone as claimed in claim 6, further comprising a temperature control element used for controlling the heating element to provide the heat energy according to the heating temperature measured by the thermometer so as to heat the ozone to a preset temperature.

8. The system for real-time monitoring of particles in ozone as claimed in claim 2, further comprising an air inlet adapter and an air outlet adapter, the air inlet adapter being connected between the air inlet conduit and the gas conveying pipe, the air outlet adapter being connected between the gas conveying pipe and the air outlet conduit.

9. The system for real-time monitoring of particles in ozone as claimed in claim 1, further comprising a cooling device for cooling the oxygen obtained by heating and reducing the ozone with the ozone reduction device.

10. The system for real-time monitoring of particles in ozone as claimed in claim 1, further comprising a process equipment, wherein the ozone source provides at least one portion of the ozone to the ozone reduction device for heating and reducing the at least one portion of the ozone into the oxygen, and the ozone source provides another portion of the ozone to the process equipment to perform a process step.

11. The system for real-time monitoring of particles in ozone as claimed in claim 10, wherein the particle counter simultaneously and instantaneously monitors the number of the particle and/or the numerical value of the particle size in the oxygen generated by heating and reducing the at least one portion of the ozone when the process equipment uses the other portion of the ozone to perform the process step.

12. The system for real-time monitoring of particles in ozone as claimed in claim 11, wherein the process equipment determines whether the ozone is contaminated by the particle based on the number of the particle and/or the numerical value of the particle size of the particle counter, thereby controlling to continue or to stop introducing the other portion of the ozone provided by the ozone source into the process equipment.

13. The system for real-time monitoring of particles in ozone as claimed in claim 10, wherein the ozone source shunts to supply the at least one portion of the ozone and the other portion of the ozone through a shunt pipe, thereby providing the at least one portion of the ozone to the ozone reduction device and providing the other portion of the ozone to the process equipment respectively.

14. The system for real-time monitoring of particles in ozone as claimed in claim 13, wherein a control valve is provided between the ozone source and the shunt pipe to control supplying or stop supplying the at least one portion of the ozone and/or the other portion of the ozone according to the number of the particle and/or the numerical value of the particle size.

15. The system for real-time monitoring of particles in ozone as claimed in claim 1, wherein the particle counter uses a light source to provide a light ray to illuminate the oxygen, causing the particle in the oxygen to scatter or diffract, and then analyzes characteristics of the light ray of the light source to obtain the number of the particle and/or the numerical value of the particle size.

16. The system for real-time monitoring of particles in ozone as claimed in claim 1, further comprising a pure oxygen source for supplying a pure oxygen to the ozone reduction device before the ozone reduction device heating the ozone provided by the ozone source to reduce the ozone into the oxygen until the number of the particle and/or the numerical value of the particle size monitored by the particle counter are/is zero.

17. A method for real-time monitoring of particles in ozone, using the system for real-time monitoring of particles in ozone as claimed in claim 1 to real-time monitor the particle in the ozone, comprising following steps:
performing an ozone providing step, for providing the ozone using the ozone source;
performing an oxidation-reduction step, for using the ozone reduction device to heat and reduce the ozone provided by the ozone source into the oxygen along the spiral conveying path; and
performing a monitoring step, for using the particle counter to monitor in real time the number of the particle and/or the numerical value of the particle size in the oxygen.

18. The method for real-time monitoring of particles in ozone as claimed in claim 17, wherein further comprising performing a zeroing step after performing the ozone providing step and before performing the oxidation-reduction step, so as to make the number of the particle and/or the numerical value of the particle size obtained by monitoring with the particle counter zero.

19. The method for real-time monitoring of particles in ozone as claimed in claim 17, wherein further comprising performing a cooling step after performing the oxidation-reduction step and before performing the monitoring step, for cooling the oxygen obtained by heating and reducing the ozone with the ozone reduction device.

20. The method for real-time monitoring of particles in ozone as claimed in claim 17, wherein further comprising performing a shunt step for shunting supply of the ozone after performing the ozone providing step and before performing the oxidation-reduction step.
